(19) United States
(12) Patent Application Publication
(10) Pub. No.: US 2005/0118261 A1
(43) Pub. Date: Jun. 2, 2005

(54) Compositions and methods of administering doxepin to mucosal tissue

(76) Inventors: Hal J. Oien, Tualatin, OR (US); Edmond Truelove, Seattle, WA (US); Joel Epstein, Wilmette, IL (US)

Correspondence Address: KOLISCH HARTWELL, P.C., 520 S.W. YAMHILL STREET, SUITE 200, PORTLAND, OR, 97204, US

(21) Appl. No.: 10/868,505

(22) Filed: Jun. 14, 2004

Related U.S. Application Data
(60) Provisional application No. 60/478,438, filed on Jun. 12, 2003.

(51) Int. Cl. A61K 31/553; A61K 9/22

(52) U.S. Cl. 424/468; 514/211.13

(57) ABSTRACT

Compositions and methods for pain relief involve delivery of doxepin to mucosal tissue. Vehicles for administering doxepin may be formulated for sustained release and/or site-specific application to maximize beneficial pain relief locally while minimizing significant adverse side effects.

### CROSS-REFERENCE TO RELATED APPLICATION

This application claims priority under 35 U.S.C. §119 from U.S. Provisional Patent Application Ser. No. 60/478,438, filed Jun. 12, 2003, which is incorporated herein by reference in its entirety for all purposes.

### FIELD

The invention relates to compositions and methods of dispensing doxepin compositions to mucosal tissue, particularly in topical vehicles for sustained pain relief.

### BACKGROUND

Doxepin is a tricyclic antidepressant drug. It is a dibenzexipin tricyclic compound (N,N-dimethyldibenz(b,e)oxepin-propylamine hydrochloride) with a formula of C₁₉H₂₁N.HCl, and has a molecular weight of 316. The action of the tricyclics appears to be both central and peripheral. The primary mechanism of action may be by affect on descending pathways by blocking reuptake of serotonin and nor-epinephrine. In the periphery, activity may relate to adenosine receptors. Doxepin has potent H1 and H2 receptor blocking activity. Recently, nonspecific enkephalin-like activity, not affecting beta-endorphins has been demonstrated in patients prescribed doxepin. The tricyclics may affect the NMDA receptor in addition to effects on the descending norepinephrine and serotinergic systems. Due to the role of NMDA-receptor-medicated effects in spinal nociception, the modulation of the NMDA receptor was studied and acetylcholine release was seen by tricyclics including doxepin, by non-competitive antagonism, suggesting that at least some of the effects of tricycles may be due to inhibition of spinal NMDA receptors, in addition to the action via monoaminergic transmission in the spinal cord.

Once systemically absorbed, doxepin is converted in the liver to desmethyldoxepin, which is an active metabolite. The metabolites are excreted in the urine following glucuronidation. Desmethyldoxepin has a half-life of 28-52 hours. Plasma levels of drug and metabolite are highly variable and correlate poorly with systemic dosing.

Systemic doxepin produces drowsiness in a significant number of patients at target plasma therapeutic ranges of plasma for treatment of depression of 30-150 ng/ml. Doxepin is contraindicated in patients with narrow angle glaucoma, or for those with urinary retention. The sedating effect of alcohol and other drugs may be potentiated by doxepin. Serious drug reactions may occur with MAO inhibitors. Cimetidine has been reported to result in higher than expected serum levels of tricyclic antidepressants (TCAs) in blood.

Doxepin is used in the management of depression and chronic pain. Systemic use leads to sleep facilitation, and pain effect, in addition to treatment of depression. Tricyclics have analgesic effects in neuropathic pain, independent of their antidepressant effect. Tricyclic antidepressants are commonly used in the management of chronic pain in low (<50 mg/day) to intermediate doses (50-150 mg/day). One review of multidisciplinary pain clinics reported use in 25% of patients with chronic pain. In another study, 36 patients with back/or neck pain and depression were treated in a placebo-controlled study and doxepin was documented to be effective in managing pain and depression. Doxepin has been used in combination with nonsteroidal analgesics in management of pain associated with advanced cancer. Systemic doxepin has been reported for use in pain management associated with stomatitis. Oral doxepin rinse has been reported to provide pain relief in patients with oral mucosal lesions due to cancer or cancer therapy. However, some patients who used an oral doxepin rinse developed adverse systemic side effects such as sedation or fatigue.

### SUMMARY

Various methods and modes of administering doxepin to relieve pain associated with mucosal tissue in a patient are described. For example, doxepin may be administered site-specifically to a mucosal region in a patient's mouth. Alternatively, doxepin may be administered topically to other mucosal tissues in other parts of the body such as ear, nose, throat, eye, genitourinary, and gastrointestinal mucosa. Doxepin may also be administered in a time-release vehicle formulated to sustain pain relief without causing significant adverse side effects such as drowsiness or sedation.

### BRIEF DESCRIPTION OF THE FIGURES

FIG. 1 is a schematic view of a delivery vehicle dispensing doxepin site-specifically to a mucosal region.

FIG. 2 is a schematic sectional view of a multi-layered doxepin delivery vehicle.

### DETAILED DESCRIPTION

Particular methods and modes of administering doxepin to mucosal tissue are described below. However, it will be appreciated that many additional formulas and manners of a administering doxepin to relieve pain associated with mucosal tissue are suggested and enabled by the description.

Doxepin may be administered site-specifically to relieve pain associated with particular mucosal regions in a patient's mouth. Doxepin may also be administered in a time-release manner to maximize its sustained effect while minimizing adverse side effects. Doxepin may be administered in a variety of vehicles such as ointment, gel, foam, film, powder, gum, lozenge, or tablet, among others. The vehicle may be atomized or formulated for dispensing in an aerosol form. Doxepin may be compounded with the vehicle in a pharmaceutically effective concentration, for example, between about 0.1% (w/w) and 5.0% (w/w). For some applications the doxepin concentration is most effective between about 0.1%(w/w) and 1.0%(w/w). Doxepin may be used to provide longer pain relief compared to topical anesthetic agents that have been used in the past. For example, some topical doxepin formulas may provide pain relief for more than 30 minutes. Other formulas may provide pain relief for even longer periods such as 1 hour, 3 hours, or more than 4 hours. Doxepin may be advantageously used for pain relief in mucosal tissues other than in the mouth, for example, ear, nose, throat, eye, genitourinary, and gastrointestinal mucosa (e.g.: foam, suppositories, etc.).

Doxepin may be incorporated advantageously in many possible vehicle forms. For example, doxepin may be incorporated in a slowly dissolving water soluble carrier strip. The strip may be applied to a target location in the mouth. The strip may be formulated to control sustained release of doxepin. The strip may be in the form of a single homogeneous sheet or film. Alternatively, the strip may have multiple layers with each layer having a different formulation, different drug compositions, different dissolution times, etc. The strip may have other ingredients such as plasticizers, flavoring agents, antimicrobial agents, adhesion components, etc. The strips/sheets may be formulated to deliver the drug primarily to the area where the strip adheres. Doxepin strips may be supplied in a continuous tape form. Doxepin dosage may be controlled or selected according to the surface area of the applied tape, or alternatively may correspond to tape thickness.

A doxepin strip or sheet, as described above, may be applied to a mucosal tissue region in an individual. The sheet may be comprised of a water soluble polymer and doxepin at a concentration sufficient to be absorbed through the mucosal tissue and to have a desired biological effect such as sustained pain relief.

The strip may be quite thin and flexible so that it dispenses doxepin in the mouth for an extended period with minimal notice or distraction to the individual. A strip may also be formulated to treat wounds such as cold sores, mucositis, or to help control post-surgical bleeding. The film may be formulated to increase or decrease adhesion to skin and mucosa. It may be adjusted by thickness and/or formulation to control the rate of dissolution. These features allow for specific vehicle designs required to place doxepin sources in specific mucosal regions and keep them there for specified amounts of time. The combination of dissolution rate, concentration of medication in the film, film size and shape all may contribute to the rate of administration. The rate can be specified and the dry film medication designed and produced to meet that specification. Films may be gamma radiation processed for sterilization as needed. Dispensing sheets may be manufactured by wet casting or extruding processes, for example, wet extruding at low temperature and pressure or dry extruding at high temperature and pressure.

Layered films may dispense unidirectionally, meaning that active ingredients are layered from the mucosa side to a neutral top layer. Alternatively, a film may dispense bidirectionally with the same or different active agents on opposite sides of the film.

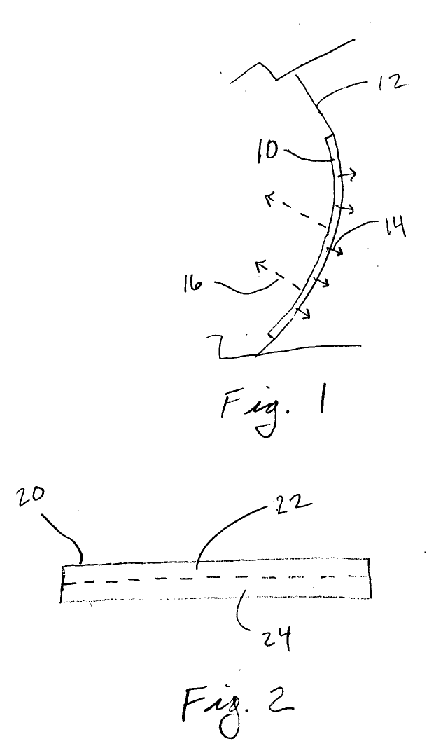

FIG. 1 shows strip 10 adhering to mucosal surface 12. Arrows 14 show the direction of doxepin permeation of mucosal tissue. Dashed line arrows 16 show the dispensing direction of the same or a different active component into the intraoral cavity. FIG. 2 shows a multilayer sheet in which layers 22 and 24 may be formulated for different purposes.

Doxepin may also be dispensed advantageously in combination with other drugs. Examples of biologically active substances that may be administered in conjunction with doxepin may include lidocaine, benzocaine, dyphenhydramine, and amitriptyline. Other topical treatments for mucosal disease (infections, reactive, autoimmune, mucositis, viral lesions, post surgical and post traumatic neuropathy, hemorrhage, stomatitis, etc.) which may be combined with doxepin include: antibiotics/antibacterials—tetracycline, chlorhexidine, metronidazol; iodine containing compounds, chlorine dioxide; antifungals—mycostatin, chlortrimazol, fluconazole, amphotericin, etc.; antivirals—acyclovir, interferon; steroids—hydrocortisone, all types and strengths of steroids, etc.; Vitamin A and other retinoids for treatment of dysplasia; azothioprine and other immune modulating medications; Tagamet—topical immune modulator; topical antineoplastic drugs—methotrexate; topical sclerosing agents; and anti-inflammatory agents. Other topical doxepin formulas may include gabapentin, clonidine, capsaicin, ginger, vitamins, buffering compounds—sodium bicarbonate, calcium, calcium carbonate, etc., coating compounds—sucrafate, eugenol, vitamin K, cocaine—hemostasis, morphine—pain control, and vitamin E.

Topical Application of doxepin with other medications for systemic absorption and effect through oral mucosa (analgesics, anxiolytics, beta blockers, nitroglycerin, hormones—estrogen etc, nicotine, sedatives and hypnotics) may include: morphine, synthetic opoid analgesics, diazepam, lorazapam, alprazolam, trialozam, propanolol, atenolol, nitroglycerin, estrogen, progesterone, testosterone, nicotine, and antihistamines.

Topical doxepin may also be compounded with one or more other analgesics, for example, acetaminophen, methyl salicylate, monoglycol salicylate, aspirin, mefenamic acid, flufenamic acid, indomethacin, diclofenac, aldlofenac, diclofenac sodium, ibuprofen, ketoprofen, naproxen, pranoprofen, fenoprofen, sulindac, fenclofenac, clidanac, flurbiprofen, fentiazac, bufexamac, piroxicam, phenylbutazone, oxyphenbutazone, clofezone, pentazocine, mepirizole, tiaramide hydrochloride, etc. Examples of steroidal anti-inflammatory agents which may be used in conjunction with doxepin include hydrocortisone, predonisolone, dexamethasone, triamcinolone acetonide, fluocinolone acetonide, hydrocortisone acetate, predonisolone acetate, methylpredonisolone, dexamethasone acetate, betamethasone, betamethasone valerate, flumetasone, fluorometholone, beclomethasone diproprionate, etc. Doxepin formulations may also include opioids for severe pain.

Cytokines or growth factors such as epidermal growth factors, or vascular-endothelial growth factors may also be included.

Doxepin may be applied to mucosal tissue in “time-release” formulations for sustained pain relief. “Time release,” as used herein, refers to “sustained release” or prolonged release of doxepin to mucosal tissue over an extended time period from a composition including doxepin and a vehicle. Accordingly, the composition may serve as a doxepin “reservoir” or “source” from which doxepin may be released gradually over the course of minutes, hours, or even days. Such gradual release may provide a sustained action of doxepin, with improved control of doxepin levels, stronger local effects, and less systemic exposure. Time release may be provided with a vehicle configured to remain substantially localized adjacent a mucosal tissue (or adjacent a selected mucosal region within the tissue) after placement of the vehicle (and doxepin) near the tissue (or region). Exemplary vehicles for time release may include solids (powders, crystals, capsules, etc.), gels, pastes, foams, viscous/sticky solutions, etc. Vehicles configured for time release may remain near the mucosal tissue/region for any suitable time period, but generally at least five or ten minutes. For some applications a vehicle is formulated to release doxepin for at least several hours.

### EXAMPLE 1

### Gels Including Doxepin

This example describes exemplary gels that may include doxepin. A gel, as used herein, is a viscous, semi-solid composition provided by a solid network holding liquid. The solid network may be a network of associated, entangled, and/or covalently linked aggregates, particles, and/or molecules, among others. Gels may be used to target extended or prolonged delivery of doxepin to a specific tissue site, such as a selected mucosal region within the mouth.

The gel may be a thixotropic gel, which is a gel that flows more readily in response to agitation and/or an applied shear stress (such as when stirred, shaken, or brushed onto a surface) and that returns to a less flowable form after the agitation and/or stress is removed. Accordingly, a thixotropic gel may have a viscosity that can be decreased before and/or during application of the gel, and that increases after application, for local retention of the applied gel. Thixotropic gels may achieve superior penetration and increased surface area contact and therefore improved uptake of doxepin. For example, thixotropic gels applied to the oral mucosa may spread until they reach a low pressure state at which point they may gel in place. This behavior may increase substantivity (longevity of clinical effectiveness) by reducing displacement of the gel by pressure.

Gels may include an amount of a gelling agent effective to form a composition for topical application. Exemplary concentrations of gelling agents are from about 0.1% to 20% by weight, or about 0.5% to 5% by weight. Gelling agents may include, among others, carboxypolymethylene, Veegum®, poloxamers, carrageenan, Irish moss, gums (such as gum karaya, gum arabic, gum tragacanth, xanthan gum, etc.), starch, alginate, polyvinylpyrrolidone, celluloses (such as hydroxyethyl propylcellulose, hydroxybutyl methyl cellulose, hydroxypropyl methyl cellulose, hydroxyethyl cellulose, carboxymethyl cellulose, carboxypropyl cellulose, and/or the like), carboxyvinyl polymers, and/or other hydroxyvinyl polymers. Other exemplary gelling agents may include Carbopol® polymers, colloidal silica, and/or complex colloidal magnesium aluminum silicates, to form thixotropic gels.

Gels may include any suitable solvents. Gels may be aqueous, that is, including water as at least the major solvent and/or the major component.

### EXAMPLE 2

### Soluble Films Including Doxepin

This example describes exemplary soluble films for dispensing doxepin. Soluble films, as used herein, are films that substantially dissolve or break down over time when disposed in contact with mucosa, and/or saliva or other bodily fluids. The films may be configured to dissolve or break down over any suitable time period, such as about thirty minutes to about twelve hours, or about two to six hours, among others.

Soluble films may be formed from any suitable composition. In some examples, soluble films may be formed by drying gels. Exemplary gels that may be dried may be formed with any suitable gel compositions listed above in Example 1.

An example of a soluble film contains doxepin at a concentration in the range of 0.1 to 5.0% (w/w). The carrier film comprises pullulan, menthol and aspartame, potassium acesulfame, copper gluconate, polysorbate 80, carrageenan, glyceryl oleate, eucalyptol, methyl alicylate, thymol, locust bean gum, propylene glycol, xanthan gum, and a coloring agent, or a subset of these components.

### EXAMPLE 3

### Foams Including Doxepin

This example describes exemplary foams including doxepin. A foam, as used herein, is a dispersion of gas bubbles in a liquid, solid, or gel. The dispersion may be stable enough to persist in a foam state for any suitable period of time, including about fifteen minutes to twelve hours, among others. Foams may be used for application to local sites.

Foams may include a solvent and various foaming agents, surfactants, emulsifiers, emulsion stabilizers, and/or foam wall thickeners, among others. Exemplary solvents may include water, an alcohol, and/or a mixture of water and an oil. Exemplary foaming agents, surfactants, emulsifiers, and/or emulsion stabilizers may include sodium lauryl sulfate, sucrose monostearate, sucrose distearate, cetyl phosphate, stearic acid, cetyl alcohol, sodium monostearate, cocoamide diethanolamine, lauramide diethanolamine, polypropylene glycol-14-butyl ether, sodium N-methyl N-cocoyl taurate, sodium methyl cocoyl-N-coco-beta-aminobutyric acid, monosodium N-lauryl-1-glutamate, and/or monosodium-N-cocoyl-1-glutamate, among others. Exemplary foam thickeners may include glycerol, sorbitol, hydrogenated starch hydrolysate, and/or the like.

Foams may be formed by any suitable mechanism. In some examples, the foams may be formed as they are dispensed from an aerosol container. Dispensing may be facilitated with an aerosol propellant, such as propane, butane, etc.

### EXAMPLE 4

### Sprays and Aerosols Including Doxepin

This example describes sprays and aerosols including doxepin. Sprays, as used herein are gas-borne solid or liquid particles, drops, and/or streams that can be directed to a surface or an area. Sprays may include particles or drops of any suitable size, generally about 10-20 micrometers or greater in diameter. Aerosols, as used herein, are fine solid or liquid particles suspended in gas. The particles in aerosols may have any suitable diameter, for example, about 1 micrometer to about 20 micrometers. Sprays and/or aerosols may permit application of doxepin to mucosal sites that are difficult to approach through other delivery mechanisms.

Sprays and/or aerosols may be formed by passing a composition including doxepin from a container through a suitable outlet structure. The outlet structure may include an atomizer, an orifice, a channel, and/or the like. The size of the drops or particles formed may be adjusted based on the size and/or shape of the outlet structure, a pressure exerted on the outlet by the composition (for example, from inside the container by a propellant in the container), the rate at which the composition is released form the container, and/or the like.

Sprays and/or aerosols including doxepin may be produced from any suitable composition. The composition may include, for example, a solvent, such as water, an additive to increase the viscosity (such as a polymer, for example, polyethylene glycol), a gelling agent, etc. Alternatively, no solvent may be included. For example, methyl cellulose can be used as a dry powder mixed with doxepin and placed in a spray container, to permit the methyl cellulose/doxepin to be directed as a powder spray from the container to selected sites.

### EXAMPLE 5

### Pastes Including Doxepin

This example describes pastes (and/or ointments or salves) including doxepin. A paste, as used herein, is a soft, plastic (moldable) composition that is semisolid. Pastes/ointments or salves may be used for application to sites of physical irritation and abrasion. A paste may be formed, for example, by mixing a suitable solvent (such as water) with a solid or a very viscous liquid. An exemplary paste for application to a mucosal tissue may be formed by mixing methyl cellulose with water. Alternatively, or in addition, a paste may include colloidal particles, such as colloidal silica, as in gel toothpastes. These colloidal pastes may be aqueous in nature and made from particles that are so small they become suspended in water without being dissolved in water.

### EXAMPLE 6

### Solid Compositions Including Doxepin

This example describes solid compositions including doxepin. Solid compositions may be suitable for application to large areas of mucosal tissue without direct contact to the mucosal tissue by the application method. Solid compositions may be in any suitable form, including a powder, crystals, pellets, capsules, etc. Doxepin may have any suitable concentration or proportion within these solid compositions, including about 0.1 to 50%, among others. The solid compositions may include any suitable vehicle, such as a simple or complex carbohydrate and/or a polymer (for example, polyethylene glycol), among others. Doxepin may be incorporated into the solid compositions by mixing, grinding, encapsulation, co-precipitation, drying a liquid or semi-solid composition, and/or the like.

Solid compositions may be applied by any suitable mechanism. Exemplary mechanisms may include mechanical application (such as with a spoon or spatula), as a powder spray, in association with an insoluble (or soluble) carrier (such as a film or tray), etc.

### EXAMPLE 7

### Insoluble Carriers and/or Barriers

This example describes insoluble carriers and/or barriers that may be used to facilitate application of doxepin to mucosal tissues. Insoluble carriers/barriers, as used herein, are structures (such as films, trays, vessels, etc.) that do not break down and/or become dispersed when exposed to saliva or other bodily fluids for a period of at least four hours. Insoluble carriers may be configured as carriers of doxepin compositions, for example, gels, pastes, foams, solutions, and/or solids including doxepin. Accordingly, these compositions may be placed on and/or in each carrier, to be held in apposition to mucosal tissue. For example, a doxepin composition may be disposed on an insoluble film. Alternatively, or in addition, the insoluble structure may function as a barrier. The barrier may restrict movement of doxepin and/or doxepin compositions away from a site of application and/or may restrict access of bodily fluids, such as salive, to the doxepin and/or the doxepin composition.

Any suitable materials may be used to form the carriers/barriers. Exemplary carriers/barriers are films formed of a plastic, such as polyethylene, polypropylene, polyvinyl chloride, a polyester, etc.

The following U.S. patent applications and patents are incorporated by reference: Ser. Nos. 10/728,277; 09/993,383; U.S. Pat. Nos. 4,517,173; 4,572,832; 4,713,243; 4,900,554; 5,137,729; 5,770,559; 5,981,474; 6,159,498; 6,479,074; 6,669,960; and 6,685,917.

1. A composition for relieving pain in a mucosal tissue region in a patient comprising
a vehicle formulated for site-specific application to a selected mucosal region in a patient's mouth, and
doxepin incorporated in the vehicle in a concentration effective for relieving pain associated with the selected mucosal region for at least about a half hour.

2. The composition of claim 1, wherein the vehicle is formulated for time release.

3. The composition of claim 1, wherein the doxepin concentration is in the range of 0.1% (w/w) to 5.0% (w/w).

4. The composition of claim 1, wherein the doxepin concentration is in the range of 0.1% (w/w) to 1.0% (w/w).

5. The composition of claim 1, wherein the concentration of doxepin is sufficient to relieve pain associated with the mucosal region for at least about one hour.

6. The composition of claim 1, wherein the concentration of doxepin is sufficient to relieve pain associated with the mucosal region for at least about three hours.

7. The composition of claim 1, wherein the vehicle is in the form of an ointment.

8. The composition of claim 1, wherein the vehicle is in the form of a gel.

9. The composition of claim 8, wherein the gel is thixotropic.

10. The composition of claim 1, wherein the vehicle is in the form of a foam.

11. The composition of claim 1, wherein the vehicle is in the form of a substantially anhydrous film.

12. The composition of claim 1, wherein the film is water soluble.

13. The composition of claim 1, wherein the vehicle is in the form of a substantially anhydrous powder.

14. The composition of claim 1, wherein the vehicle is in an atomized form.

15. The composition of claim 1, wherein the vehicle is in an aerosol form.

16. The composition of claim 1, wherein the mucosal region is in the patient's mouth.

17. The composition of claim 1, wherein the mucosal region is ocular

18. The composition of claim 1, wherein the composition is formulated for administration to a human patient.

19. The composition of claim 1, wherein the composition is formulated for veterinary administration.

20. A composition for relieving pain in a patient comprising
a vehicle formulated for time-release administration of an active ingredient to treat a mucosal region in a patient, and
doxepin incorporated in the vehicle in a concentration effective for relieving pain associated with the mucosal region for at least about a half hour.

21. The composition of claim 20, wherein the composition is formulated for site-specific application in a patient's mouth.

22. The composition of claim 20, wherein the doxepin concentration is in the range of 0.1% (w/w) to 5.0% (w/w).

23. The composition of claim 20, wherein the doxepin concentration is in the range of 0.1% (w/w) to 1.0% (w/w).

24. The composition of claim 20, wherein the concentration of doxepin is sufficient to relieve pain associated with the mucosal region for at least about one hour.

25. The composition of claim 20, wherein the concentration of doxepin is sufficient to relieve pain associated with the mucosal region for at least about three hours.

26. The composition of claim 20, wherein the vehicle is in an aqueous form.

27. The composition of claim 20, wherein the vehicle is substantially anhydrous.

28. The composition of claim 20, wherein the vehicle comprises PEG.

29. The composition of claim 20, wherein the vehicle is in the form of chewing gum.

30. The composition of claim 20, wherein the vehicle is in the form of a lozenge.

31. The composition of claim 20, wherein the vehicle is in the form of a soluble tablet.

32. The composition of claim 20, wherein the vehicle is in the form of a gel.

33. The composition of claim 32, wherein the gel is thixotropic.

34. The composition of claim 20, wherein the vehicle is in the form of an ointment.

35. The composition of claim 20, wherein the vehicle is in the form of a foam.

36. The composition of claim 20, wherein the vehicle is in the form of a substantially anhydrous film.

37. The composition of claim 20, wherein the film is water soluble.

38. The composition of claim 20, wherein the vehicle is in the form of a substantially anhydrous powder.

39. The composition of claim 20, wherein the vehicle is in an atomized form.

40. The composition of claim 20, wherein the vehicle is in an aerosol form.

41. The composition of claim 20, wherein the mucosal region is in the patient's mouth.

42. The composition of claim 20, wherein the mucosal region is ocular.

43. The composition of claim 20, wherein the composition is formulated for administration to a human patient.

44. The composition of claim 20, wherein the composition is formulated for veterinary administration.

45. A method of relieving pain in a patient comprising
providing a composition comprising doxepin in an effective concentration and suitable vehicle for site-specific relief of pain associated with a selected mucosal region in a patient's mouth, and
administering the composition site-specifically to the mucosal region.

46. The method of claim 45, wherein the vehicle is formulated for time release.

47. A method of relieving pain in a patient comprising
providing a composition comprising doxepin in an effective concentration and suitable time-release vehicle for relief of pain associated with a selected mucosal region on a patient, and
administering the composition to the mucosal region.

48. The method of claim 47, wherein the administering step includes applying the composition site-specifically in the patient's mouth.

49. A composition for relieving pain in a patient comprising
a vehicle formulated for administration of an active ingredient to treat an ocular mucosal region in a patient, and
doxepin incorporated in the vehicle in a concentration effective for relieving pain associated with the mucosal region for at least about a half hour.

50. The composition of claim 49, wherein the vehicle is aqueous.

51. The composition of claim 49, wherein the vehicle is formulated for time release of doxepin.

52. The composition of claim 49, wherein the vehicle is formulated for dispensing as eye drops.

53. A method of relieving pain associated with a mucosal region in a patent comprising
providing a composition comprising doxepin in an effective concentration and suitable vehicle for relief of pain associated with an ocular mucosal region in the patient, and
administering the composition to the mucosal region.

54. The method of claim 53, wherein the administering step includes dispensing eye drops into the patient's eye.

55. The method of claim 53, wherein the administering step includes spraying the composition on the mucosal region.
